(19) United States
(12) Patent Application Publication
(10) Pub. No.: US 2019/0133832 A1
(43) Pub. Date: May 9, 2019

(54) Absorbent Adhesive Nipple Cover

(71) Applicants: Heather Langley, (US); David Langley, (US)

(72) Inventors: Heather Langley, (US); David Langley, (US)

(21) Appl. No.: 15/804,857

(22) Filed: Nov. 6, 2017

(51) Int. Cl. A61F 13/14

(52) U.S. Cl. CPC A61F 13/141

(57) ABSTRACT

An absorbent, self-adhesive nipple cover for absorbing fluids from a lactating woman's nipple while concealing the nipple and areola in such a way so as to flatter the appearance of the woman's breast for intimate wear or to disguise that the woman is nursing. The inner layer, facing the woman's breast, is comprised of two sections: a central section made of absorbent material, and a perimeter section made of one or more adhesive appendages that secure the nipple cover completely to the woman's breast. The outer layer, facing away from the woman's breast, is comprised of protective aesthetic material to prevent fluids from leaking through the absorbent material onto clothes or other materials that may come into contact with the nipple cover, and to promote the appearance of the woman's breasts while concealing the nipple and areola.

### CROSS-REFERENCE TO RELATED APPLICATIONS (11)

| Publication Number | Priority Date | Publication Date | Assignee | Title |
|---|---|---|---|---|
| U.S. Pat. No. 6,036,577A | 1998 Feb. 18 | 2000 Mar. 14 | Shonda L. Coburn | Disposable Breast Pad |
| US20120129428A1 | 2010 Oct. 12 | 2012 May 24 | Zaleatra Keiaunna Taylor | Adhesive Nursing Pad |
| U.S. Pat. No. 6,039,629A | 1999 May 6 | 2000 Mar. 21 | Julia Mitchell | Nursing Pad |
| U.S. Pat. No. 5,782,672A | 1996 May 28 | 1998 Jul. 21 | Vickie G. Woodley | Nipple Pad |
| U.S. Pat. No. 4,333,471A | 1981 Jan. 19 | 1982 Jun. 8 | Tokiwa Chemical Ind Ltd | Nipple Cover |
| U.S. Pat. No. 5,326,305A | 1992 Sep. 10 | 1994 Jul. 5 | Fochler Zhou Li | Protective Breast Pad |
| U.S. Pat. No. 5,690,536A | 1995 Dec. 20 | 1997 Nov. 25 | Kathleen A. D. Madden | Brassiere Liner |
| US20090088717A1 | 2007 Sep. 28 | 2009 Apr. 2 | Charles J. Pawloski, Tina M. Bruce | Absorbent Nursing Pad Adhesive Cover |
| U.S. Pat. No. 5,017,174A | 1990 Jan. 18 | 1991 May 21 | Felicia B. Gowrylow | Nursing Pad |
| U.S. Pat. No. 4,047,534A | 1977 Jan. 3 | 1977 Sep. 13 | Kimberly-Clark Corp | Nursing Pad |
| US20060057938A1 | 2004 Sep. 10 | 2006 Mar. 16 | Pei-Hui Huang | Nipple Stick With Absorption Layer |

### BACKGROUND OF THE INVENTION

### 1. Field of the Invention

The present invention relates to absorbent pads and milk breast pads, and more particularly to a self-adhesive, absorbent nipple cover with aesthetic qualities.

### Classifications:

A61F13/141 Milk breast pads

A41C3/065 Strapless brassieres, i.e. without shoulderstraps attached directly to the body, e.g. by means of adhesive

A61F2013/15016 Absorbent pads, e.g. sanitary towels, swabs or tampons for external or internal application to the body; Supporting or fastening means therefor; Tampon applicators characterized by the use for breast; pads for bras

### 2. Description of the Related Art

Typically, nursing and pregnant mothers will absorb their leaking milk with nursing pads that are supported with the use of brassieres, which can be confining in normal wear, restrictive in intimacy, and prohibitive when wearing certain types of revealing clothing. For example, see U.S. Pat. No. 4,047,534 to Kimberly-Clark Corp; U.S. Pat. No. 5,017,174 to Gowrylow; U.S. Pat. No. 6,039,629 to Mitchell.

Attempts have been made to create nursing pads with adhesive material, but these attempts are intended to entirely cover the woman's breasts, or to give added support as brassieres, or they fail to promote intimate wear, or they are confined to certain shapes, sizes or curvatures, or they do not flatter the woman's breasts or otherwise disguise that the woman is a nursing or pregnant mother. For examples, see U.S. patent application Ser. No. 13/200,308, Publication No. 20120129428A1 (Published May 24, 2012) (Zaleatra Keiaunna Taylor, applicant); U.S. patent application Ser. No. 12/240,339, Publication No. 20090088717A1 (Published Apr. 2, 2009) (Charles J. Pawloski, Tina M. Bruce, applicants); U.S. patent application Ser. No. 11/161,732, Publication No. 20060057938A1 (Published Mar. 16, 2006) (Pei-Hui Huang, applicant).

Other attempts have been made to create self-adhesive nipple covers for intimate wear and to flatter a woman's breasts, but these attempts do not provide absorbable padding for leaking milk, nor do they promote intimacy specifically for nursing and pregnant mothers who require absorbent protection from staining clothing or other contacted materials. For example, see U.S. Pat. No. 5,782,672 to Woodley; U.S. Pat. No. 4,333,471 to Nakai.

Lastly, attempts have been made to create adhesive breast pads that eliminate the requirement of a brassiere but have neither an absorbent function nor are they intended to be worn as intimate wear. For example, see U.S. Pat. No. 6,036,577 to Coburn; U.S. Pat. No. 5,326,305 to Fochler.

Consequently, there is a great need for nursing and pregnant mothers who want to flatter their breasts (for intimate purposes or for wearing certain types of revealing clothing) and/or so that sexual partners can be uninhibited in foreplay, all the while to still be afforded a form of absorbent protection for when they are lactating. The present invention addresses this need.

### BRIEF SUMMARY OF THE INVENTION

A primary object of the present invention is to facilitate intimacy for nursing and pregnant women who, while lactating, are otherwise confined to wearing a combination of devices like brassieres and nursing pads that act as obstacles to intimacy.

Another object of the present invention is to absorb milk from the nipples of nursing and pregnant women without prohibiting the aforementioned intimacy.

Another object of the present invention is to conceal the nipple and areola of nursing and pregnant women's breasts in such a way so as to flatter the natural appearance of her breasts.

A further object is to provide an absorbent pad for nursing and pregnant women that adheres to the contour of the breast so as to promote the touch and feel of the breast.

Yet another object of the present invention is to provide an absorbent device for nursing and pregnant women that can disguise that the woman is nursing when wearing certain types of revealing clothing.

These and other objects and advantages of the present invention will become apparent from the following description taken in conjunction with the accompanying drawings, wherein is set forth an embodiment of this invention.

### BRIEF DESCRIPTION OF THE SEVERAL VIEWS OF THE DRAWING(S)

FIG. 1 is a perspective view of the inner layer of the present invention, with two sections: a central absorbent material, and one or more adhesive appendages that secure the nipple cover completely to the woman's breast.

FIG. 2 is a perspective view of the layers of the present invention: outer layer of material that conceals the nipple and areola; a central absorbent material; and an inner adhesive layer.

FIG. 3 is a perspective view of the outer layer of the present invention with various appearances, all performing the same function.

FIG. 4 is a perspective view of the outer layer of the present invention that conceals the nipple and areola.

### DETAILED DESCRIPTION OF THE INVENTION

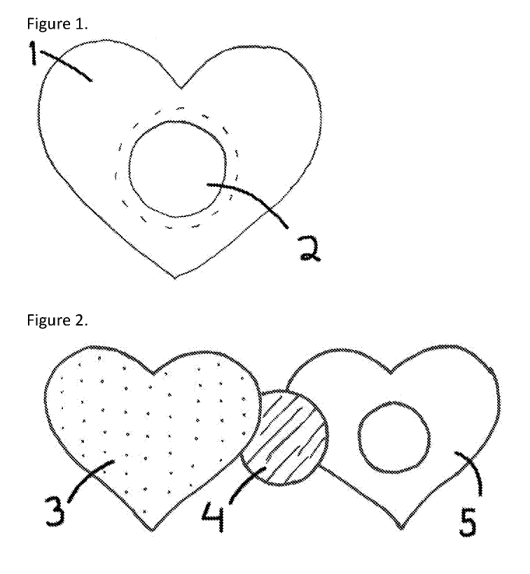

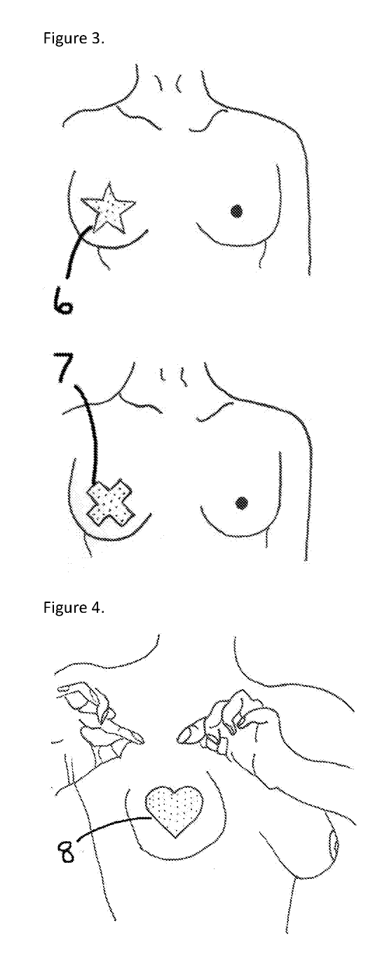

With reference now to the embodiments, as illustrated in FIGS. 1-3, an absorbent adhesive nipple cover of the present invention comprises an inner layer with one or more adhesive appendages 1, a central absorbent material 2, and an outer layer of aesthetic material 3, wherein the adhesive appendage(s) 1 can be of varying shapes (including but not limited to a heart-shape 8, a cross 7, or a star 6) that secure the nipple cover completely to the woman's breast, and eliminate the need for a brassiere or other types of support devices; a central absorbent material 4 that makes direct contact with a nursing or pregnant woman's nipple in order to absorb leaking milk from her breast without the use of a traditional nursing pad or other devices that may absorb or restrict the flow of milk, and to prevent fluids from leaking through the absorbent material onto clothes or other materials that may come into contact with the nipple cover; and an outer layer of protective aesthetic material 3 that prevents fluids from leaking through the absorbent material onto clothes or other materials while concealing the nipple and areola 6 in such a way so as to flatter the appearance of the woman's breasts and to promote intimacy with a sexual partner. The outer layer 3 may also potentially help disguise that a woman is nursing when wearing certain types of revealing clothing. The outer layer is also aesthetically-attractive and made of material that adheres to the contour of the woman's breast so as to flatter the breast's appearance and to promote the breast's touch and feel.

The absorbent adhesive nipple cover of the present invention as described above, unlike other support devices and nursing apparel, facilitates intimacy for nursing and pregnant women who, while lactating, are otherwise likely confined to wearing a combination of devices like brassieres and nursing pads that act as obstacles to intimacy. The present invention promotes the natural appearance of a woman, flatters the appearance of her breasts, and promotes intimacy, all the while achieving protection for periods of lactation and freeing the woman from the confinement of other protective devices. In addition, the present invention promotes feelings of confidence and happiness with one's self.

1. An absorbent, self-adhesive nipple cover for absorbing fluids from a lactating woman's nipple while concealing the nipple and areola. The nipple cover is comprising of:
a) an inner layer, facing the woman's breast, with a central absorbent pad and a perimeter with adhesive material;
b) an outer layer, facing away from the woman's breast, comprised of material for aesthetic qualities and for protection from leakage through the inner layer to the outer layer of the nipple cover

2. The absorbent pad of claim 1a is a material for absorbing fluids leaking from the woman's nipple, and which is completely surrounded by the adhesive material around the perimeter of the inner layer.

3. The adhesive material of claim 1a is around the perimeter of the inner layer, and secures the nipple cover completely to the woman's breast.

4. The outer layer of claim 1b is of aesthetic qualities that promotes the appearance of the woman's breasts while concealing the nipple and areola.

5. The outer layer of claim 1b is comprised of protective material to prevent fluids from leaking through the absorbent material of claim 1a onto clothes of other objects or materials that may come into contact with the nipple cover.
